(19) United States
(12) Patent Application Publication
(10) Pub. No.: US 2011/0092768 A1
(43) Pub. Date: Apr. 21, 2011

(54) DEVICE, SYSTEM AND METHOD FOR IN-VIVO ANALYSIS

(75) Inventors: Noam Emanuel, Jerusalem (IL); Emil-Israel Katz, Savyon (IL); Elena Khazanov, Beit Shemesh (IL); Abraham Rubinstein, Jerusalem (IL); Yechezkel Barenholz, Jerusalem (IL); Abdel Kareem Azab, Ara (IL)

(73) Assignee: GIVEN IMAGING LTD., Yoqneam (IL)

(21) Appl. No.: 12/740,764

(22) PCT Filed: Nov. 2, 2008
(86) PCT No.: PCT/IL2008/001435

§ 371 Date: Oct. 20, 2010

Related U.S. Application Data
(60) Provisional application No. 61/001,104, filed on Oct. 31, 2007.

(51) Int. Cl. A61B 1/04; B05D 1/36; B05D 5/00

(52) U.S. Cl. 600/109; 427/2.11

(57) ABSTRACT

A device for in-vivo detection comprises a housing having an optical window and enclosing an imager that is configured to image the optical window. An external surface of the optical window has trypsin immobilized thereon, and may also be coated with a steric barrier protection, which may be polyethylene glycol (PEG). A trypsin-Alpha-1-antitrypsin complex formed on the window may have an affinity to a binding agent, which is tagged by a tag selected from a group consisting of a colorant, a fluorescent moiety, and a radioactive moiety.

### FIELD OF THE INVENTION

The present invention relates to in-vivo analysis in general, and to in vivo analysis using swallowable capsules in particular.

### BACKGROUND OF THE INVENTION

An atypical concentration or presence of substances in body fluids or in body lumens may be indicative of the biological condition of the body. For example, the presence of elevated concentrations of red blood cells in the gastrointestinal (GI) tract may indicate different pathologies, depending on the location of the bleeding along the GI tract. Thus, for example, bleeding in the stomach may indicate an ulcer, whereas bleeding in the small intestine may indicate the presence of a tumor. Furthermore, different organs may contain different body fluids requiring different analysis methods. For example, the stomach secretes acids whereas pancreatic juice is basic.

Alpha-1-antitrypsin (A1AT), which is a serine protease inhibitor and trypsin inhibitor, typically protects tissues from enzymatic degradation and is normally present in the blood. However, high level of alpha-1-antitrypsin in gastric juice has been found to be strongly associated with gastric cancer.

Medical detection kits are usually based on in vitro testing of body fluid samples for the presence of a suspected substance. For example, in some cases, diseases, such as cancer, are detected by analyzing the blood stream for tumor specific markers, typically, specific antibodies. A drawback of this method is that the appearance of antibodies in the blood stream usually occurs at a late stage of the disease, such that early detection is not possible using this method. Furthermore, some molecules may normally appear in the blood but may indicate pathology when present in other organs or body fluids.

Early detection, identification and location of abnormal conditions (such as, for example, an atypical presence or concentration of a substance) may be critical for definitive diagnosis and/or treating of various pathologies.

Devices, systems and methods for in-vivo sensing of passages or cavities within a body, and for sensing and gathering information (e.g., image information, pH information, temperature information, electrical impedance information, pressure information, etc.), are known in the art.

Swallowable imaging capsules can sample intestinal fluids to a chamber within the capsule while traversing the GI tract and may perform analysis of the sample in the chamber for the presence of suspected substances onboard the capsule.

### SUMMARY OF THE INVENTION

Various embodiments of the invention provide devices, systems and methods of in-vivo analysis. Embodiments of the invention enable in vivo analysis of body lumen fluids for the presence of substances, for example, markers for cancer.

Embodiments of the invention include the use of a first binding agent and a second binding agent. Both binding agents may have an affinity for the same marker which may be present in the body lumen environment (for example, endo-luminal fluids).

According to one embodiment the first binding agent is immobilized to an in vivo sensing device such that when the device is introduced in vivo the first binding agent is exposed to the body lumen environment and may bind the marker, if the marker is present in the environment.

The second binding agent is tagged, for example, by adding to it a colorant, a fluorescent moiety, a radioactive moiety or any other suitable tag. According to some embodiments the second binding agent may be bound to the surface of tagged particles. The second binding agent may also be protected and stabilized by the attachment of a polymer such as polyethylene glycol (PEG) or any other naïve molecule. According to an embodiment of the invention the second binding agent may be separately introduced into the endo-luminal environment so that it may bind the marker or the first binding agent/marker complex. Thus, if a marker is present in the endo-luminal environment it will bind to the first binding agent on the sensing device and then the second tagged binding agent will bind the bound marker thereby highlighting the presence of the marker.

According to one embodiment the first binding agent may be trypsin, the marker to be detected may be A1AT and the second binding agent may be an antibody to A1AT. According to other embodiments other tumor, inflammation or other pathology markers may be targeted. Examples of such markers may include collagen (and denaturized collagen) (which may indicate open areas in a damaged tissues), fibrin cloth and albumin (that may indicate recent bleeding) angiogenic factors (that may indicate tumors and other abnormal conditions based on, for example, their concentration and/or the location) The in vivo imaging device may be an imaging device or any other suitable sensor.

By using different markers simultaneously the understanding of the actual pathological state of a patient may be enriched.

According to some embodiments the in vivo sensing device includes a housing configured to be inserted in vivo and a sensor contained within the housing. In some embodiments, for example, the in-vivo device may include a transmitter to transmit data from the in-vivo sensor.

In some embodiments, for example, the in-vivo device may include a housing having a substantially transparent portion, such as an optical window, and an imager that is able to acquire an image through the transparent housing portion.

In some embodiments, for example, the imager is to acquire an in-vivo image of a body lumen, typically of the GI tract.

In some embodiments, for example, a system may include the in-vivo device, an external receiver/recorder able to receive data (e.g., image data) transmitted by the in-vivo device, and a computing platform or workstation able to store, process, display, or analyze the received data.

According to one embodiment of the invention a first binding agent is attached to a surface configured to be inserted in vivo. For example, a first binding agent may be attached to the external surface of an optical window of a capsule endoscope. In this case, the surface immobilized binding agent can be attached as a monolayer, or as a multilayer. The multilayer composition can be composed of a polymer or macromolecule backbone, and several binding agents can be attached at different location along the chains. Alternatively, different attachment locations may be exploited in different polymers or macromolecules. This multilayer structure can allow binding of more than one layer of the tagged second binding agent, thus elevating the resulted signal.

According to one embodiment a surface coating may be added for stabilized and enhanced attachment of the first binding agent and to reduce non specific binding to the surface, and by that to increase signal-to-noise ratio.

According to another embodiment of the invention the first binding agent is a free tagged molecule or is attached to a labeled particle. According to one embodiment tagged binding agents can be pre-stabilized by the attachment of protecting molecules such as polyethylene glycol, thereby increasing their stability and specificity.

A method according to one embodiment of the invention may include the step of attaching a binding agent (such as trypsin or any other suitable substrate or binding agent as well as suitable antibodies and/or antibody fragments) onto an optical window of a capsule endoscope. The method may include a complementary step of coating the external surface of the optical window with suitable material, for example, polyethylene glycol (PEG), polymer that is attached at one end to the surface of the optical window, to reduce non-specific binding of molecules to the optical window surface.

According to embodiments of the invention a method for in vivo analysis is provided. According to one embodiment the method for in vivo analysis may include the steps of: introducing an in vivo sensing device having a first binding agent attached to it; administering a tagged second binding agent; and receiving a reading from the in vivo sensing device. According to one embodiment the method of analysis includes the step of elevating the stomach pH. According to some embodiments the step includes raising the stomach pH to a level of between approximately 5.5 and 7.4. According to some embodiments this step may include administering acid reducing agents.

A kit for in vivo analysis is further provided according to one embodiment of the invention. The kit may include a second binding agent that is tagged directly or a binding agent that attached to a non-modified or a tagged particle, with or without a steric barrier protection, typically in a solution and an acid reducing buffer reagent. In another embodiment the kit may include a first tagged binding agent, with or without a complementary second tagged binding agent, both with or without a steric barrier protection. Components of the kit may be taken by a patient as part of a screening procedure which may also include being administered, for example, by a physician, a device according to embodiments of the invention. Alternatively, a kit may include a capsule endoscope having immobilized thereon a first binding particle for self administration and a second binding particle. Optionally an acid reducing buffer agent may be included in the kit.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of an in vivo detecting system according to one embodiment of the invention;

FIGS. 2A-C are schematic illustrations of an in vivo sensing device according to embodiments of the invention;

FIG. 3 is a schematic diagram of a method according to an embodiment of the invention;

FIG. 4 is a schematic diagram of a method of in vivo analysis according to one embodiment of the invention

FIG. 5 is a schematic diagram of measured affinity between several antibodies and a marker according to one embodiment of the invention;

FIG. 6 is a schematic diagram of a time dependent interaction between an antibody and a marker according to one embodiment of the invention;

FIG. 7 is a schematic diagram of measured affinity between a second antibody and a first antibody/marker complex according to one embodiment of the invention;

FIG. 8 is a schematic diagram of measured affinity between an antibody and a marker in different pH levels according to one embodiment of the invention; and

FIG. 9 is a schematic diagram of measured affinity between an antibody and a marker in different pH levels according to another embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

It should be noted that although a portion of the discussion may relate to in-vivo imaging devices, systems, and methods, the present invention is not limited in this regard, and embodiments of the present invention may be used in conjunction with various other in-vivo sensing devices, systems, and methods. For example, some embodiments of the invention may be used, for example, in conjunction with in-vivo sensing of pH, in-vivo sensing of temperature, in-vivo sensing of pressure, in-vivo sensing of electrical currents, in-vivo detection of a substance or a material and/or various other in-vivo sensing devices, systems, and methods. Some embodiments of the invention may be used not necessarily in the context of in-vivo imaging or in-vivo sensing.

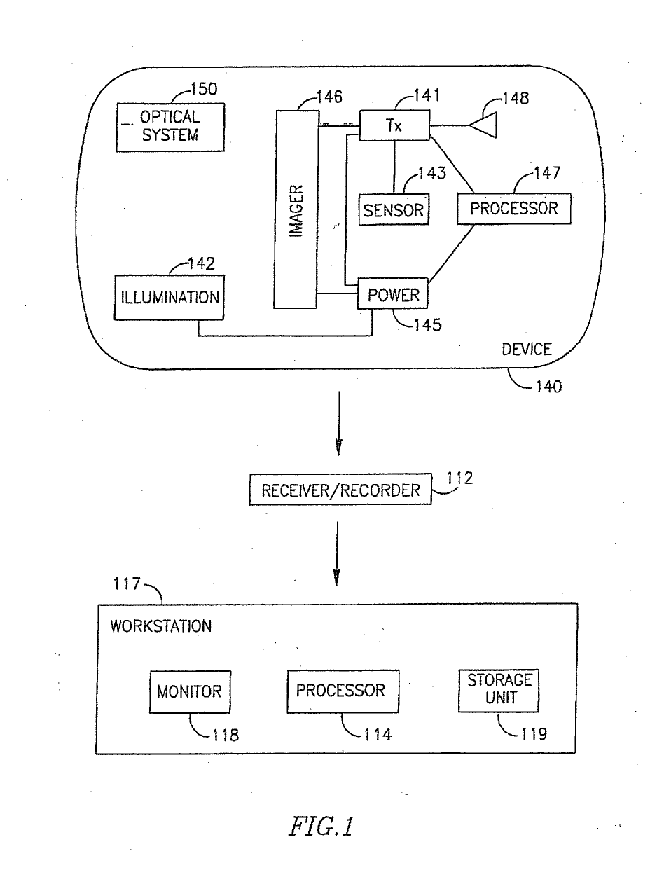

Some embodiments of the present invention are directed to a typically swallowable in-vivo sensing device, e.g., a capsule endoscope. Devices according to embodiments of the present invention may be similar to embodiments described in U.S. Pat. No. 7,009,634, entitled “Device And System For In-vivo Imaging”, filed on 8 Mar., 2001, and/or in U.S. Pat. No. 5,604,531 to Iddan et al., entitled “In-vivo Video Camera System”, and/or in International Application number WO 02/054932 entitled “System and Method for Wide Field Imaging of Body Lumens” published on Jul. 18, 2002, all of which are hereby incorporated by reference. An external receiving unit and processor, such as in a work station, such as those described in the above publications could be suitable for use with embodiments of the present invention. Devices and systems as described herein may have other configurations and/or other sets of components. For example, the present invention may be practiced using an endoscope, needle, stent, catheter, etc. Reference is now made to FIG. 1, which schematically illustrates a system according to an embodiment of the invention. In some embodiments, the system may include a device 140 having a sensor, e.g., an imager 146, one or more illumination sources 142, a power source 145, and a transmitter 141. In some embodiments, device 140 may be implemented using a swallowable capsule, but other sorts of devices or suitable implementations may be used. Outside a patient's body may be, for example, an external receiver/recorder 112 (including, or operatively associated with, for example, one or more antennas, or an antenna array), a storage unit 119, a processor 114, and a monitor 118. In some embodiments, for example, processor 114, storage unit 119 and/or monitor 118 may be implemented as a workstation 117, e.g., a computer or a computing platform.

Transmitter 141 may operate using radio waves; but in some embodiments, such as those where device 140 is or is included within an endoscope, transmitter 141 may transmit/receive data via, for example, wire, optical fiber and/or other suitable methods. Other known wireless methods of transmission may be used. Transmitter 141 may include, for example, a transmitter module or sub-unit and a receiver module or sub-unit, or an integrated transceiver or transmitter-receiver.

Embodiments of device 140 are typically autonomous, and are typically self-contained. For example, device 140 may be a capsule or other unit where all the components are substantially contained within a housing or shell, and where device 140 does not require any external wires or cables to, for example, receive power or transmit information. In some embodiments, device 140 may be autonomous and non-remote-controllable; in another embodiment, device 140 may be partially or entirely remote-controllable. In some embodiments, device 140 may communicate with an external receiving and display system (e.g., workstation 117 or monitor 118) to provide display of data, control, or other functions. For example, power may be provided to device 140 using an internal battery, an internal power source, or a wireless system able to receive power. Other embodiments may have other configurations and capabilities. For example, components may be distributed over multiple sites or units, and control information or other information may be received from an external source.

In some embodiments, device 140 may include an in-vivo video camera, for example, imager 146, which may capture and transmit images of, for example, the GI tract while device 140 passes through the GI lumen. Other lumens and/or body cavities may be imaged and/or sensed by device 140. In some embodiments, imager 146 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In some embodiments, imager 146 in device 140 may be operationally connected to transmitter 141. Transmitter 141 may transmit images to, for example, external transceiver or receiver/recorder 112 (e.g., through one or more antennas), which may send the data to processor 114 and/or to storage unit 119. Transmitter 141 may also include control capability, although control capability may be included in a separate component, e.g., processor 147. Transmitter 141 may include any suitable transmitter able to transmit image data, other sensed data, and/or other data (e.g., control data) to a receiving device. Transmitter 141 may also be capable of receiving signals/commands, for example from an external transceiver. For example, in some embodiments, transmitter 141 may include an ultra low power Radio Frequency (RF) high bandwidth transmitter, possibly provided in Chip Scale Package (CSP).

In some embodiment, transmitter 141 may transmit/receive via antenna 148. Transmitter 141 and/or another unit in device 140, e.g., a controller or processor 147, may include control capability, for example, one or more control modules, processing module, circuitry and/or functionality for controlling device 140, for controlling the operational mode or settings of device 140, and/or for performing control operations or processing operations within device 140. According to some embodiments, transmitter 141 may include a receiver which may receive signals (e.g., from outside the patient's body), for example, through antenna 148 or through a different antenna or receiving element. According to some embodiments, signals or data may be received by a separate receiving device in device 140.

Power source 145 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo device 140.

In some embodiments, power source 145 may be internal to device 140, and/or may not require coupling to an external power source, e.g., to receive power. Power source 145 may provide power to one or more components of device 140 continuously, substantially continuously, or in a non-discrete manner or timing, or in a periodic manner, an intermittent manner, or an otherwise non-continuous manner. In some embodiments, power source 145 may provide power to one or more components of device 140, for example, not necessarily upon-demand, or not necessarily upon a triggering event or an external activation. Optionally, in some embodiments, transmitter 141 may include a processing unit or processor or controller, for example, to process signals and/or data generated by imager 146. In another embodiment, the processing unit may be implemented using a separate component within device 140, e.g., controller or processor 147, or may be implemented as an integral part of imager 146, transmitter 141, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In some embodiments, for example, the processing unit or controller may be embedded in or integrated with transmitter 141, and may be implemented, for example, using an ASIC.

In some embodiments, imager 146 may acquire in-vivo images continuously, substantially continuously, or in a non-discrete manner, for example, not necessarily upon-demand, or not necessarily upon a triggering event.

In some embodiments, transmitter 141 may transmit image data continuously, or substantially continuously, for example, not necessarily upon-demand, or not necessarily upon a triggering event.

In some embodiments, device 140 may include one or more illumination sources 142, for example one or more Light Emitting Diodes (LEDs), “white LEDs”, or other suitable light sources. Illumination sources 142 may, for example, illuminate a body lumen or cavity being imaged and/or sensed. An optional optical system 150, including, for example, one or more optical elements, such as one or more lenses or composite lens assemblies, one or more suitable optical filters, or any other suitable optical elements, may optionally be included in device 140 and may aid in focusing reflected light onto imager 146, focusing illuminated light, and/or performing other light processing operations.

In some embodiments, illumination source(s) 142 may illuminate continuously, or substantially continuously, for example, not necessarily upon-demand, or not necessarily upon a triggering event. In some embodiments, for example, illumination source(s) 142 may illuminate a pre-defined number of times per second (e.g., two or four times), substantially continuously, e.g., for a time period of two hours, four hours, eight hours, or the like; or in a periodic manner, an intermittent manner, or an otherwise non-continuous manner. In some embodiments, the components of device 140 may be enclosed within a housing or shell, e.g., capsule-shaped, oval, or having other suitable shapes. The housing or shell may be substantially transparent or semi-transparent, and/or may include one or more portions, windows or domes which may be substantially transparent or semi-transparent. For example, one or more illumination source(s) 142 within device 140 may illuminate a body lumen through a transparent or semi-transparent portion, window or dome; and light reflected from the body lumen may enter the device 140, for example, through the same transparent or semi-transparent portion, window or dome, or, optionally, through another transparent or semi-transparent portion, window or dome, and may be received by optical system 150 and/or imager 146. In some embodiments, for example, optical system 150 and/or imager 146 may receive light, reflected from a body lumen, through the same window or dome through which illumination source(s) 142 illuminate the body lumen.

Data processor 114 may analyze the data received via external receiver/recorder 112 from device 140, and may be in communication with storage unit 119, e.g., transferring frame data to and from storage unit 119. Data processor 114 may provide the analyzed data to monitor 118, where a user (e.g., a physician) may view or otherwise use the data. In some embodiments, data processor 114 may be configured for real time processing and/or for post processing to be performed and/or viewed at a later time. In the case that control capability (e.g., delay, timing, etc) is external to device 140, a suitable external device (such as, for example, data processor 114 or external receiver/recorder 112 having a transmitter or transceiver) may transmit one or more control signals to device 140.

Monitor 118 may include, for example, one or more screens, monitors, or suitable display units. Monitor 118, for example, may display one or more images or a stream of images captured and/or transmitted by device 140, e.g., images of the GI tract or of other imaged body lumen or cavity. Additionally or alternatively, monitor 118 may display, for example, control data, location or position data (e.g., data describing or indicating the location or the relative location of device 140), orientation data, and various other suitable data. In some embodiments, for example, both an image and its position (e.g., relative to the body lumen being imaged) or location may be presented using monitor 118 and/or may be stored using storage unit 119. Other systems and methods of storing and/or displaying collected image data and/or other data may be used.

Typically, the image data recorded and transmitted may include digital color image data; in alternate embodiments, other image formats (e.g., black and white image data) may be used. In some embodiments, each frame of image data may include 256 rows, each row may include 256 pixels, and each pixel may include data for color and brightness according to known methods. According to other embodiments a 320×320 pixel imager may be used. Pixel size may be between 5 to 6 micron. According to some embodiments pixels may be each fitted with a micro lens. For example, a Bayer color filter may be applied. Other suitable data formats may be used, and other suitable numbers or types of rows, columns, arrays, pixels, sub-pixels, boxes, super-pixels and/or colors may be used.

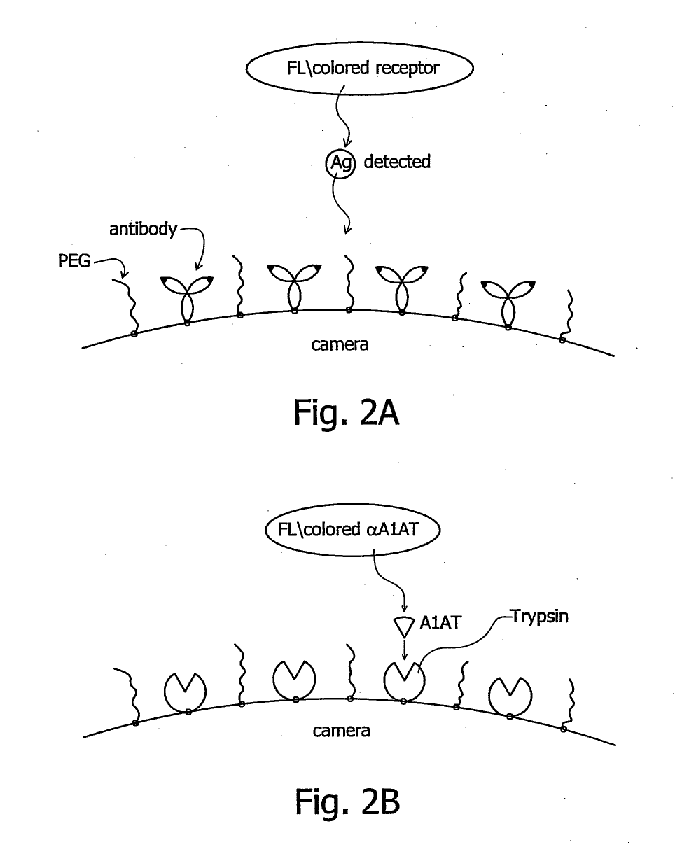

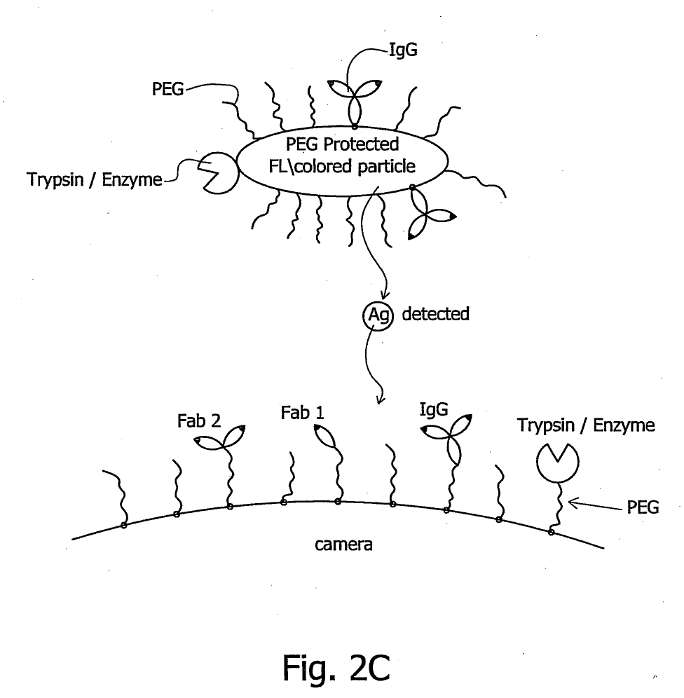

Optionally, device 140 may include one or more sensors 143, instead of or in addition to a sensor such as imager 146. Sensor 143 may, for example, sense, detect, determine and/or measure one or more values of properties or characteristics of the surrounding of device 140. For example, sensor 143 may include a pH sensor, a temperature sensor, an electrical conductivity sensor, a pressure sensor, or any other known suitable in-vivo sensor. Reference is now made to FIGS. 2A-C which schematically illustrate a device according to several embodiments of the invention.

According to an embodiment of the invention the in vivo sensing device is a capsule endoscope. The capsule endoscope typically has a dome shaped optical window at one or both ends of the capsule. Other windows are possible, for example the optical window may be along a side of the device or surrounding the device. Behind the optical window, enclosed within the capsule housing are positioned an image sensor or other light receptor, an optical system for focusing images onto the image sensor and at least one illumination source for illuminating the GI tract through which the capsule endoscope is propagating.

According to one embodiment a binding agent is adhered to the optical window of the capsule endoscope. The binding agent may bind a marker prevalent in the GI tract lumen. The binding agent/marker complex may then bind a second binding agent which contains a color or other tag. In the case that the second binding agent binds to the complex on the optical window, the colored binding agent will be in the field of view of the image sensor and may appear as a colored spot or other shaped mark in an image being obtained by the image sensor.

According to one embodiment the in vivo sensing device may include a sensor such as a sensor of electrical charge to sense a change in electrical charge which may indicate a change in the configuration of the first binding agent due to its interaction with the marker.

According to one embodiment, for example as illustrated in FIG. 2A, the external surface of an optical window is coated. Typically the optical window is made of a plastic such as Isoplast® or polycarbonate. Other solid phase substrates may be used, for example, glass, silica, or other plastics, such as polypropylene and polystyrene. Sometimes, surface characteristics of the substrate may affect immobilization or coupling of peptide or protein antigens or antibodies. To avoid this effect a surface coating may be used such as PEG and its derivatives or other naïve molecules such as albumins. The coating may include molecules having a molecular weight adjusted to that of the first binding agent which for one-sided attachment of PEG polymers, for example, will normally range from 1,000-10,000 Dalton.

A first binding agent may then be adhered to the optical window. The first binding agent may be an antibody or its fragments (Fab2 or Fab, or single-chain antibodies) having a suitable affinity to the marker. The marker may be a GI tract cancer marker such as CEA or CA 19-9. For example, a system of monoclonal antibodies directed against different antigenic determinants on CA 19-9 may be used. Other antibodies may be used, for example, anti-TNF alpha monoclonal antibodies may be used in the detection of Crohn's disease, as well as a natural or recombinant soluble/membrane TNF binding agent. Antibodies to other known GI tract cancer markers or other pathologies may be used.

Once the coated in vivo device is introduced into the GI tract (for example, by swallowing) the antibody immobilized onto the optical window may come into the vicinity of a marker, if that marker is present in the GI tract. The marker will then bind to the antigen forming a complex on the optical window. Typically, the surface coating and the bound antibody and/or complex are transparent in the wavelengths used for illumination by the in vivo device. Thus the in vivo device may image the GI tract unobstructed.

A second binding agent, for example, a second antibody, may be introduced into the GI tract (for example, by any appropriate method of administration). The second antibody, which typically, but not necessarily, has an affinity to a different antigenic determinant on the marker or on the complex, also has a detectable moiety, such as a color bead, a fluorescent moiety, a radioactive moiety, a magnetic bead, gold particles as well as other metal colloidal particles or other appropriate detectable agent. In the case where a marker binds to the first antibody thus being immobilized to the optical window, the second antibody will bind to the bound marker (or to the first binding agent/marker complex) and will thus also be immobilized on the optical window. Since the second antibody includes a colorant or other detectable moiety, the presence of the bound second antibody may be detected, either by being viewed and imaged by the image sensor of the capsule endoscope or by other suitable detecting means which may be included in the capsule endoscope, for example, other optical detectors or a radiation detector.

Data sensed by the in vivo device according to embodiments of the invention, may be transmitted to an external receiver and may be viewed and/or analyzed by a processor out side the body. Data sense by the device, for example, image data, may include indication of the presence of the second binding agent. The presence of the second binding agent may be indicative of the presence of the marker in the lumen being examined and as such may indicate to a physician that the patient being examined may be in danger of developing cancer or other pathologies.

According to another embodiment illustrated in FIG. 2B, the external surface of an optical window is coated, for example by PEG and a first binding agent, for example, trypsin or other protease such as pepsin, chemotrypsin, elastase, is immobilized onto the optical window. When in vivo, the bound trypsin (as an example) may come into the vicinity of its inhibitor A1AT, which is also a marker for gastric cancer. A1AT from the GI tract fluids may bind to the trypsin on the optical window and thus the A1AT itself may be immobilized onto the optical window. The second binding agent used in this case may include a tagged antibody for A1AT/trypsin complex. According to other embodiments the first binding agent and the second binding agent may include the same molecules. For example, the first binding agent may include pepsin, (or chemotrypsin, elastase, trypsin or any other relevant protease) and the second binding agent may include a colored or tagged pepsin (or chemotrypsin, elastase, trypsin or any other relevant protease) binding agent. The tagged antibody or tagged trypsin will bind the immobilized A1AT and will thus be detected by the capsule endoscope. According to another embodiment illustrated in FIG. 2 C an in vivo sensing device may include two or more types of binding agents, for example to enhance binding of the desired marker or to enable detection of a plurality of different markers.

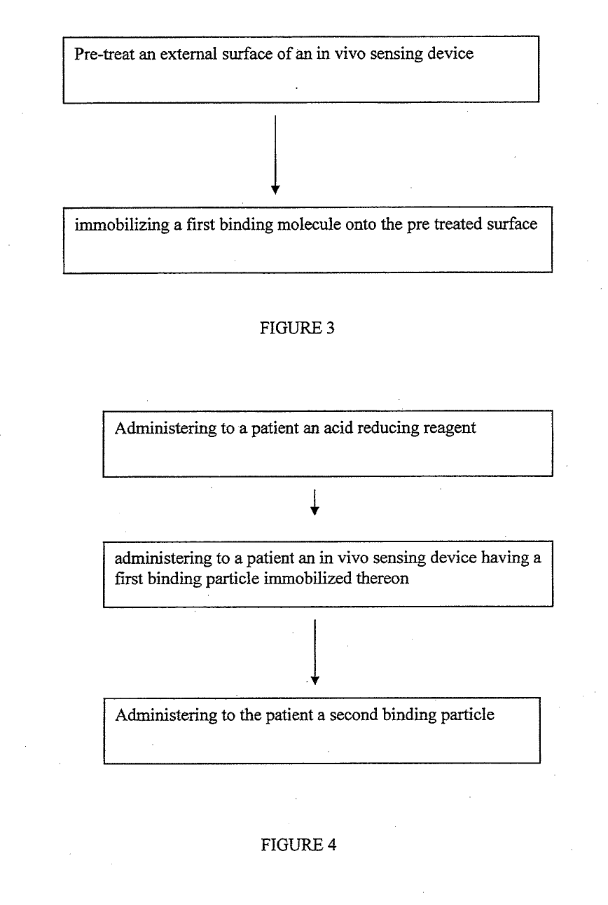

Reference is now made to FIG. 3, which schematically illustrates a method according to an embodiment of the invention. According to one embodiment the method includes the steps of immobilizing a first binding agent molecule onto an external surface of an in vivo sensing device. The first binding agent may typically be a peptide or protein, carbohydrate and may be immobilized by known methods of immobilizing peptides or proteins or other molecules to surfaces, for example, plastic or silica surfaces. The immobilization of the binding agent to a support depends on the specific characteristics of both the binding agent and the support. According to one embodiment the binding agent may be applied directly to the support such as in the immobilizing of poly electrolytes onto the support. According to another embodiment the binding agent may be applied onto a modified support, to a pretreated support or the binding agent may be immobilized to the support via a bridging group. Other methods of immobilization are possible.

An optional step according to one embodiment includes the attachment of steric barrier molecules to the external surface of the in vivo device, such as by coating the surface with PEG.

According to one embodiment the first binding agent may be adhered to an optical window of a capsule endoscope. The window is typically within the field of view of an image sensor contained within the capsule. The binding agent may be bound to specified areas of the window, such as to a ring on a dome shaped window or to corners of other shaped windows. Alternatively, binding agents may be adhered to substantially the whole window area. Following is an exemplary protocol used to detect free alpha-1-antitrypsin precursors in buffers and biological fluids. This example is in no way intended to limit the scope of the invention.
1. Coating—Plates (96 wells, flat bottom, treated to gain high protein absorbance) were coated with Trypsin (from bovine pancreas) by the addition of 50 μl of 10 μg protein in phosphate buffer saline (PBS) pH=7.0 supplemented with sodium azide (0.025% w/w) as a preservative to each well. Typically the plates were incubated for 60 min at 37° C.
2. Blocking—The plates were washed two times with 250 μl/well of wash solution (PBS supplemented with sodium azide and nonionic detergent Tween-20 at final concentration of 0.05%). Subsequently, a PBS supplemented with 1% (w/w) of bovine serum albumin (BSA) and sodium azide (0.025% w/w) were added at a final volume of 200 μl/well, and incubated for 60 min at 37° C. At the end of the incubation the wells are washed 3 times by using 250 μl of wash solution/well (ambient temperature).
3. Samples—The inspected samples were added to the plate at a final volume of 50 μl/well, and typically serially diluted by using the relevant diluter (example: for human plasma samples the dilutor may be human α₁-antitrypsine precursor (A1AT) negative plasma from rabbit). The plates were incubated for 60 min at 37° C. subsequently the wells were washed 3 times with a wash buffer (ambient temperature).
4. Antibody I—Antibody directed to human A1AT (anti A1AT) was added to the wells, typically in 50 μl/well of PBS supplemented by sodium azide and BSA as described in step No 2. The plates were incubated for 60 min at 37° C. and subsequently washed 3 times with a wash buffer (ambient temperature).
5. Antibody (II) conjugate—Antibody directed to the relevant isotype of antibody I and conjugated to horse radish peroxidase (HRP conjugate) is added to the wells, typically in 50 μl/well of washed buffer (but other A1AT samples are also relevant) are incubated for 60 min at 37° C. and subsequently washed 5 times with a wash buffer (ambient temperature).
6. Substrate—TMB reagent, the HRP substrate, is added in citrate buffer (pH=5) supplemented with peroxides at a final volume of 100 μl/well.
7. Stop reaction—When sufficient yellow coloration appears the reaction is stopped by the addition of 1M H2SO4 at a final volume of 100 μl/well.

Reference is now made to FIG. 4, which illustrates a method of in vivo analysis according to one embodiment of the invention. According to one embodiment the method includes the steps of administering to a patient a device according to embodiments of the invention and administering to the patient a second binding agent. The second binding agent may be in a solution including pharmaceutically acceptable additives. According to other embodiments the second binding agent may be in any other suitable form, such as in a powder, spray or suspension.

Administering a device in vivo may be done in any suitable way such by swallowing by the patient or otherwise inserting the device into the patient's GI tract.

The timing of the different administrations may be planned such to allow sufficient time for the first binding agent to bind the marker and only then for the marker-first binding agent complex to bind the tagged second binding agent.

According to another embodiment of the invention the first binding agent is a free tagged molecule or a binding agent that is attached to a labeled particle. For example, the first binding agent may be attached to its target marker and can be directly viewed or otherwise detected from the optical window of a capsule endoscope. In another example, one fluorescently tagged binding agent may be attached to its target marker side by side with a complementary fluorescently tagged binding agent, resulting in a combined active fluorescent emission that can be detected by the optical detector (such as an imager) of, for example, a capsule endoscope. According to one embodiment the tagged binding agents can also be pre-stabilized by the attachment of molecules such as polyethylene glycol, improving their stability and specificity to their ligand molecules.

According to one embodiment an acid reducing agent may be administered to the patient. Acid reducing agents, such as known antacids (e.g., Maalox, Rolaids etc.) will typically raise and buffer the pH level in the stomach, thus providing a more stable environment for the binding agents (typically proteins) and for the markers themselves. For example, acid reducing agents may neutralize pepsin in the stomach and may inhibit the activation of protease precursors that are secreted from the pancreas into the bowel, thus providing an environment essentially free of active pepsin for the procedure of the invention. According to one embodiment a pH level of between about 6.0 to about 7.4 may be desirable. According to one embodiment pH in the range of 6-8 is optimal for stable trypsin (as well as other relevant proteases that can bind A1AT)/A1AT complex formation. However, other pH levels may also be obtained according to embodiments of the present invention. For example, according to one embodiment a pH of above 5.5 may be obtained.

Embodiments of the present invention provide a novel in vivo screening procedure and a novel use of A1AT in an in vivo screening procedure for cancer in the GI tract, for example, gastric cancer.

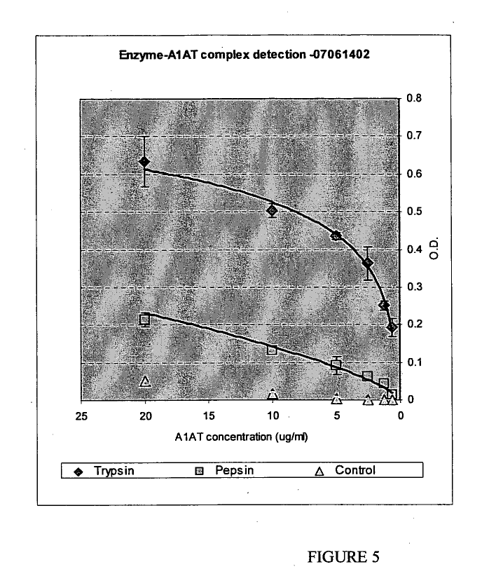

Reference is now made to FIG. 5 which is a schematic diagram of measured affinity between several antibodies and a marker according to one embodiment of the invention. Plates (96 wells, flat bottom, treated to gain high protein absorbance) were coated with Trypsin or Pepsin which are the molecules to bind to a marker. In the control plates there was no enzyme coating, however bovine serum albumin (BSA) was used to wash the control wells along with the Trypsin and Pepsin coated wells in order to avoid non specific interaction of proteins with the wells surface. In this embodiment, the marker, a human α₁-antitrypsin precursor (A1AT), was serially diluted and allowed to interact with all coated and non-coated wells. All wells were washed and polyclonal anti-A1AT was added as the second antibody of the reaction. In some embodiments, in order to view the binding between Trypsin/Pepsin and the marker human α₁-antitrypsin precursor (A1AT), another antibody conjugated to horse radish peroxidase (HRP) is added to the wells. In this embodiment, a goat anti-rabbit IgG conjugated to HRP was added to the wells. The A1AT concentration was calculated from the Optical density (O.D.) of each set of wells (i.e., Trypsin, Pepsin and control). It can be inferred from the diagram that the highest concentration of A1AT found in the wells was in the wells coated with Trypsin. This shows a high affinity between Trypsin and A1AT, which indicated Trypsin may be a good binding agent to be used when screening for A1AT as a marker for gastric cancer.

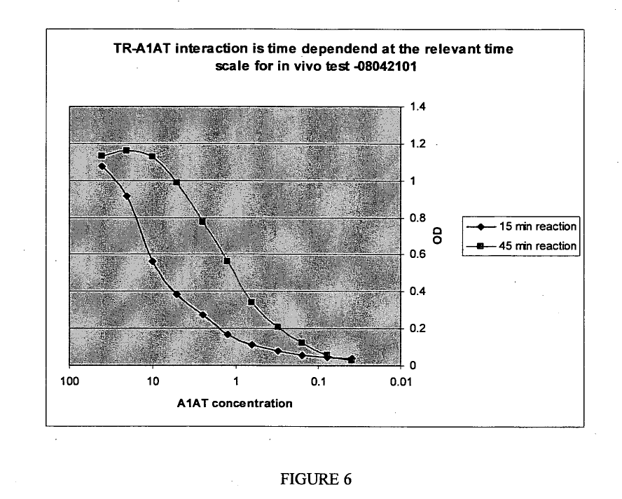

Reference is now made to FIG. 6 which is a schematic diagram of a time dependent interaction between an antibody and a marker according to one embodiment of the invention. Plates (96 wells, flat bottom, treated to gain high protein absorbance) were coated with Trypsin. Human α₁-antitrypsin precursor (A1AT) which is the marker, was serially diluted and allowed to interact with the immobilized Trypsin for either 15 minutes or 45 minutes. The two different sets of wells were then washed and polyclonal anti-A1AT was added as the second antibody of the reaction. In some embodiments, in order to view the binding between Trypsin and the marker which is the human α₁-antitrypsin precursor (A1AT), another antibody conjugated to horse radish peroxidase (HRP) is added to the wells. In this embodiment, a goat anti-rabbit IgG conjugated to HRP was added to the wells. The diagram of FIG. 6 shows that for a 15 minutes time reaction between A1AT and Trypsin as well as for a 45 minutes time reaction between A1AT and Trypsin, an optical density (O.D.) signal is acquired.

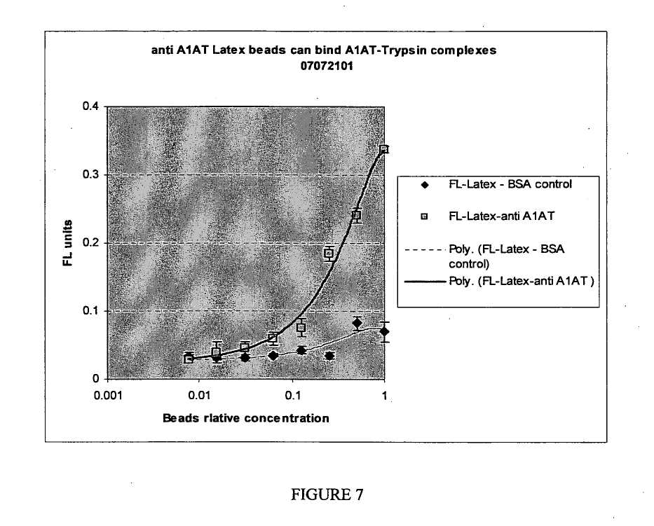

In some embodiments, the binding agent which may be Trypsin, is coated on an external surface of an optical window/dome of a capsule endoscope. In some embodiments, after the in-vivo imaging device is swallowed it passes along the esophagus and then reaches the stomach. Such an in-vivo imaging device may stay in the stomach for an average time of 15 minutes. And so, according to this embodiment, those 15 minutes are enough to acquire a signal showing binding between Trypsin and A1AT, which is a marker for gastric cancer. Reference is now made to FIG. 7 which is a schematic diagram of measured affinity between a second antibody and a first antibody(marker)/substrate complex according to one embodiment of the invention. In this example, Plates (96 wells, flat bottom, treated to gain high protein absorbance) were coated with A1AT/Trypsin complex. In this example, fluorescently tagged 100 nm latex beads were attached to Rabbit anti-A1AT polyclonal antibody. In this embodiment, the Rabbit anti-A1AT polyclonal antibody with the latex beads was incubated with the A1AT/Trypsin complex and washed so unbound anti-A1AT polyclonal antibody with beads would not be present. A control was also prepared by using fluorescently tagged latex beads attached to bovine serum albumin (BSA). In this example, the fluorescently tagged beads had a peak of excitation of 360 nm and a peak of emission of 420 nm. Measurements were taken in both the A1AT/Trypsin complex wells and the control wells, by using excitation wavelength of 360 nm and emission wavelength of 460 nm. The diagram of FIG. 7 shows that fluorescence intensity grows in correlation to the growing concentration of beads, and in addition that the fluorescence intensity of the beads attached to the A1AT/Trypsin complex is greater than the fluorescence intensity of the beads attached to the control group. This may indicate the benefit of using fluorescently tagged beads attached to a second antibody (i.e. the Rabbit anti-A1AT polyclonal antibody) so as to indicate the presence in-vivo of A1AT as a marker for gastric cancer.

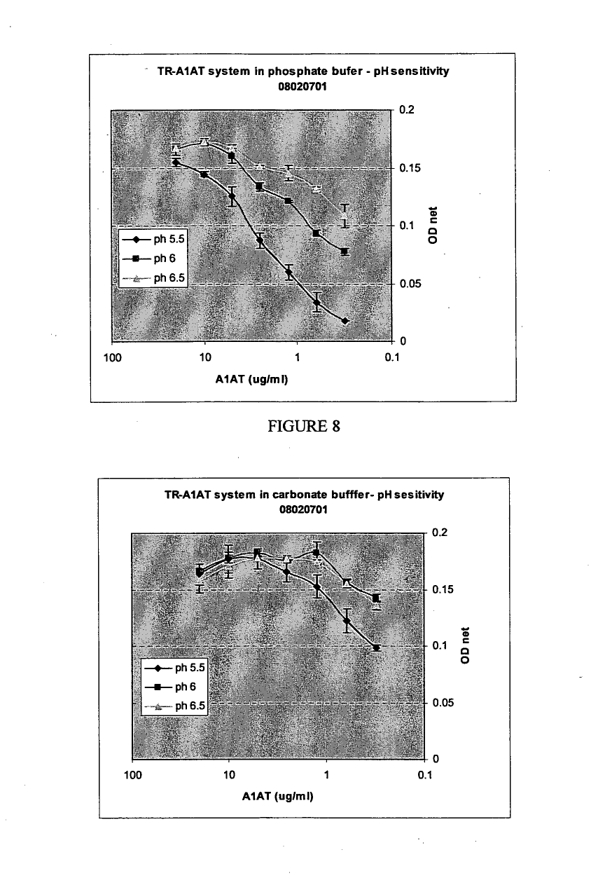

Reference is now made to FIGS. 8 and 9. FIG. 8 is a schematic diagram of measured affinity between an antibody and a substrate in different pH levels according to one embodiment of the invention, and FIG. 9 is a schematic diagram of measured affinity between an antibody and a substrate in different pH levels according to another embodiment of the invention.

In these examples, the affinity between Trypsin and A1AT was measured in different pH levels (e.g. pH 5.5, pH 6 and pH 6.5), in two types of buffers. In FIG. 8 the affinity between Trypsin and A1AT was measured in the presence of phosphate buffer, and in FIG. 9, the affinity between Trypsin and A1AT was measured in the presence of carbonate buffer. From comparing FIGS. 8 and 9, it can be inferred that the affinity between Trypsin and A1AT is less sensitive to its pH environment when carried out in carbonate buffer than when carried out in phosphate buffer. This may indicate on carbonate buffer as a better buffer to use when screening for A1AT as a marker for gastric cancer, since in the presence of carbonate buffer the changes in-vivo in pH less effect the binding between Trypsin and A1AT marker. While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A device for in-vivo detection, the device comprising:
a housing said housing comprising an optical window and said housing enclosing an imager;
wherein an external surface of the optical window has a steric barrier protection coated thereon and a binding agent immobilized thereon, and wherein the imager is configured to image the optical window.

2. The device according to claim 1, wherein said steric barrier protection is polyethylene glycol (PEG).

3. A system for in-vivo detection, the system comprising:
an in vivo sensing device comprising:
a housing said housing comprising an optical window and said housing enclosing an imager;
wherein an external surface of the optical window has a steric barrier protection coated thereon and a binding agent immobilized thereon, and wherein the imager is configured to image the optical window; and
a transmitter to transmit images from the imager;
a receiving system to receive transmitted signals; and
a display to display indication of the presence of a marker in vivo.

4. The system according to claim 3, wherein said coated steric barrier protection is polyethylene glycol (PEG).

5. A method for manufacturing an in-vivo detection device, the method comprising:
immobilizing a binding agent onto an external surface of an optical window of an in-vivo sensing device; and
coating the external surface of the optical window with a steric barrier protection.

6. The method according to claim 5, wherein said steric barrier protection is polyethylene glycol (PEG).

7. A method for in-vivo detection, the method comprising:
administering an in vivo sensing device having a window, said window coated with steric barrier protection, said window having a binding agent immobilized to it, wherein said binding agent binds to an in-vivo marker to form an immobilized complex;
detecting an indication of a reaction between said in-vivo marker and said binding agent; and
receiving a reading of the indication from the in vivo sensing device.

8. The method according to claim 7, wherein said steric barrier protection is polyethylene glycol (PEG).

9. The method according to claim 7 wherein detecting is by imaging the window.
